(19) United States
(12) Patent Application Publication
(10) Pub. No.: US 2014/0120620 A1
(43) Pub. Date: May 1, 2014

(54) Culturing solution and method of using the same, which solution is used for in-vitro support of the growth of fertilized oocytes though embryonic development to the implantation stage

(71) Applicants: Michael D. Cecchi, Madison, CT (US); Monica Mezezi, Guelph (CA)

(72) Inventors: Michael D. Cecchi, Madison, CT (US); Monica Mezezi, Guelph (CA)

(21) Appl. No.: 13/694,097

(22) Filed: Oct. 26, 2012

(51) Int. Cl. C12N 5/02

(52) U.S. Cl. CPC C12N 5/0604
USPC 435/404; 435/325

(57) ABSTRACT

This invention relates to a media solution, which has been shown to be a stabilized balance of growth media, which may contain a combination of proteins, insulin, growth hormones, and/or steroids to assist an embryo to grow from the retrieval stage through the return implantation. The media has shown consistent, improved results and overall embryonic outcome and may be used for culturing the embryos through each stage of growth, will reduce shock and stress to the embryos during the culturing process.

This application claims the benefit of U.S. Ser. No. 61/628,278, filed Oct. 28, 2011

### TECHNICAL FIELD

This invention relates to a media solution, which has been shown to be a stabilized balance of growth media, which may contain a combination of proteins, insulin, growth hormones, and/or steroids to assist an embryo to grow from the retrieval stage through the return implantation. The media has shown consistent, improved results and overall embryonic outcome and may be used for culturing the embryos through each stage of growth, will reduce shock and stress to the embryos during the culturing process.

### BACKGROUND ART

Current culture media used the area of in-vitro culturing encounter problems or limitation on through ability to properly feed or nourish the embryo during all its growth stages, through to the implantation into the patient's body. The problem facing these solution are firstly, the ingredients are not properly balanced to support growth, the solutions may be used in sequence and therefore be absolutely different solutions, and the addition of proteins, such as human serum albumen and synthetic proteins, growth factors, steroids and insulin to these media solution result in a distortion of the ingredient concentrations and therefore may actually promote negative impact on the embryo.

The problems faced by the media's used in embryo development is the factors of consistency and their ability to produce good embryos, which result in their ultimate pregnancy and eventual live birth.

Current media manufacturers change the formulation for the different changes in embryonic development and may therefore impact the embryo through these changes. The embryos may be moved through at least two different media solutions with overall ingredients solution or balance of ingredients. These media represent the stages, at least the first stage of embryo development from the beginning of culturing to the cleavage stage, of about 3 days or the 4 cell stage. The second stage is embryonic development from the cleavage stage to the blastocyst stage or 8 eight cell stage, or approximately day 5 or 6. The current philosophy of changing the media compositions for the each stage allows little flexibility as to when the actual embryo transfer occurs. The fact that they may not be feed the full balance of ingredients will hamper their growth in the early stages and may be require a longer culture time and therefore limits the time of which the embryo may be implanted. The second problem facing a media composition, which is to sustain the embryonic needs, is the addition to the media of proteins, synthetic proteins, insulin, human growth factors and other additives. The addition of these various ingredients either administered individually or together will destabilize the media and decrease the results and negatively impact the embryonic development.

Another problem of the use of at least two media solutions to provide the nutrients at the various stages of development are that they have very significant differences which makes each media solution a completely separate and different products. There are absolute and significant differences, such as, contain several different ingredients in the formulation, they have separately weighed and mixed formulations, have different overall composition, each batch manufactured separately and therefore are tested separately and may have various results from these tests. The significant tests they need to pass are mouse embryo assay testing, endotoxin testing, sterility testing, shelf life testing. The materials will likely be stored and shipped separately and may be at different times.

When using most of the conventional products or procedures for the development of oocytes through the blastocyst stage may require several solutions, one for retrieval, one for fertilization, one for development from day one to day 3, one for development from day 3 to day 5 or 6 and then one for the transfer. In this example the process calls for the use of up to 5 individual solutions, some other conventional processes may require up to 7 or 8 solutions used in sequence. The inventions allow the process to be the use of a single solution for all these process steps as well as the ability to use a single bottle of solution for all stages.

Another common practice is for the end users to add additional ingredients to the base media solutions, as or before they use it. Currently most user will add, proteins, insulin, growth factors, which will be purchased separately and at different times. The practice of adding these ingredients means different manufacturing dates, different manufactures controls, and more importantly various compositions are based on the accuracy of the individual(s) performing the task of allocating. These inconsistencies drastically increase the likelihood of embryonic problems and the reduced implantation rates, a reduction in pregnancy rates and reduced live birth rates.

An additional problem faced by the first and second stage media solutions, either used as is or those of which an end user adds some addition ingredients or additives is this will strongly increase the likelihood that they have different osmolality, different pH levels, as they are now totally distinctively different solutions from what they began with, and will likely result in adverse impact and effect on the specific embryos.

The manufacturing of different batches of separate solution and either using them ‘as is’ or to add ingredients later greatly increase the chances of error. This increases the number of variables from each composition to the actual desired ingredient amounts and the resulting outcome of pH (pH is the measure of the acidity or basicity of an aqueous solution) testing, endotoxicity testing, sterility testing and the actual shelf life, means that some 10 to 30 variables now needs to be considered in the reliability of each solution.

The currently trend is for most end users to use or make a different media formulation and solution for each phase of development. These media solutions also need additional manufacturing and being different batches. This method increases the number of ingredients and the creation of different overall solutions. This means using a media solution plus the additives such as protein will be different for the first stage and a totally different media solution for the second stage.

It would be desirable to be able to provide embryos with a more consistent and reliable solution and culture media to provide a consistent solution, ingredients, the same solution over the entire development process, while providing all the needed ingredients for all stages of embryonic growth.

### DISCLOSURE OF THE INVENTION

This invention relates to an improved media solution for embryonic development for human reproduction and other culturing needs. The invention provides the embryos with a total and complete solution, which will not require any addition ingredients. Use of the solution of the invention drastically decreases the imputed problems of embryonic development by strategically removing the variables. The solution of the invention may be used from the day one through to the blastocyst, and allow the end user to use one bottle of the media for the entire growth of the embryo, as the media include the correct overall ingredients for growth, the correct balance of proteins, synthetic proteins, insulin, human growth factors or steroids.

Thus the user may use the same bottle of media solution throughout the entire process. This means that the user cans aliquot the solution for the development of the embryo from day one to day eight without having to add any additional ingredients. Current products require that the media be changed out during the process, which requires at least two separate media bottles of different formulations and may not have the same amount of other ingredients such as salts, bicarbonates, vitamins and other ingredients usual to culture media, but also the variations of additives, such as proteins and human growth hormones, to carry the development to the desired implantation stage.

The invention allows the user to use an ingredient full, single media solution through the stages of embryonic development, without having to supplement them with any proteins, while insuring consistency and outcome.

The invention allows the end user to use an ingredient complete, single media solution through the stages of embryonic development, without having to supplement, before or during use, with any steroids, growth factors, and/or insulin while insuring consistency and outcome.

One advantage of this invention is that it allows the end user the ability to implant the embryo at any interval in the growth pattern. The embryo during culturing will make significant changes each day of development. The embryo develops in a span of from day one to day 5 or 6. If taken to the blastocyst stage and then may be implanted at Day 5 or 6. If the user decides to implant when utilizing other media which are deficient in some ingredients they may need to wait to a latter stage or add ingredients, not necessarily added to the media, but must be done to compensate for the media deficiency. This is also important if the user deemed it necessary to implant the embryo due to other reasons, in advance of their original intentions.

As the invention includes all the ingredients deemed necessary for growth and is used by the user from day one, the user may implant the embryo at any day along the way, knowing that the embryo have been fed all the needed ingredients, as will be evident in the visual evaluation of the embryo. This becomes significant, if for any reason the embryo needs to be transferred before anticipated, such as a power outage, impending weather conditions or medical reasons.

This means that the media solution has given the embryos all the needed nutrients, each step of development and therefore is confident the embryo is capable of being implanted into the patient, on day one, or day two, or day three, or day four, or day five or day six or any partial day count.

The invention allows proteins and other additive to be added, accurately equilibrated with the full range of ingredients intact, with consistent and repeatable balance of desired ingredients.

Our studies have proven the media solution to outperform the products, which are used in sequence, where the proteins are later, added, where steroids, human growth factors are added as used. The media solution pH should be about 7.35, and an osmolality of about 265.

The invention contains a complex blend of ingredients which has shown superior performance when compared to conventional solutions, an example of the ingredient concentration in millimeter per liter are:

| Ingredient | Range | Ingredient | Range |
|---|---|---|---|
| D-Glucose | 0.1-0.3 | EDTA (diNa) | 0.005-0.02 |
| KCl | 1.5-2.5 | CaCl₂•2H20 | 1.5-3.0 |
| KH₂PO. | 0.05-0.5 | Lactate (Na salt)* | 9.0-11.0 |
| MgSO. | 0.01-0.3 | NaCl | 80.0-105.0 |
| NaHCO₃ | 15.0-30.0 | Sodium Pyruvate | 0.1-0.3 |
| Glycine | 0.01-0.09 | L-Lysine HCl | 0.05-0.2.5 |
| L-Alanine | 0.01-0.09 | L-Arginine HCl | 0.1-0.3 |
| L-Asparagine | 0.1-0.3 | L-Aspartic acid | 0.03-0.07 |
| L-Cystine | 0.05-0.09 | L-Glutamic Acid | 0.05 |
| glycyl-glutamine | 0.2-.020 | L-Histidine HCl. | 0.01-0.10 |
| L-Isoleucine | 0.05-0.2 | L-Leucine | 0.05-0.35 |
| L-Methionine | 0.01-0.09 | L-Phenylalanine | 0.01-0.10 |
| L-Proline | 0.01-0.10 | L-Serine | 0.01-0.20 |
| L-Treonine | 0.05-0.5 | L-Tryptophan | 0.002-0.030 |
| L-Tryosine | 0.05-0.10 | L-Valine | 0.05-0.6 |
| Gentamicin | 0.001-0.010 | Phenol Red | 0.05-5.0 |

These ingredients will consist of about 10%, by volume, of the protein ingredients.

An additional advantage of this invention is the ability of the user to use a single solution throughout the entire process. Including the essential protein to the ingredients creates a single solution, which is critical in the development of oocytes and embryos. The solution and its formulation create a single culture solution for the development of embryos after retrieval from a female ovary. The solution is a single culture solution which can be used for the development of immature oocytes, during the fertilization stage, during the development to the four-cell stage, and then during the development to the eight-cell, or blastocyst stage. This culture solution is unique in its composition and is a departure from conventional culturing solutions and current related protocols that are used to culture oocytes and embryos through all these steps and stages.

The media may include additional ingredients to aid the embryos in overcoming certain conditions, such as their inability to expand or develop to a blastocyst stage. For these reasons the solution may include insulin, human growth factors or steroids. These ingredients have shown the ability to provide improvements in embryonic development.

The solution of this invention has proven to provide improved performance and will result in better embryonic development when using particular protein combinations. These combinations include human serum albumin, alpha globulin, and beta globulin. A preferred combination of these ingredients is a forty four milligrams of human serum albumin and about three milligrams each of alpha globulin and beta globulin, making up a fifty milligram solution. The solution may use a human serum albumin component which makes up about ninety nine percent of the protein source.

The solution may include growth factors, such as G-CSF and GM-CSF which help certain embryos in their development.

Most of the current media solution and protocols utilize several media solutions, used in sequence to accomplish that, which is attainable by this single solution. The inclusion of the needed proteins, insulin, steroids or growth factors, either individually or in combination results in the fact that the users do not need to add anything to the solution for it to accomplish these tasks.

An example of one of the conventional protocols would include at least five different products, one for each of the following stages: embryo collection; fertilization; first stage for cleavage; second stage for blastocyst stage; and then a transfer media. Each of these products would have been manufactured separately, at different times, which would result in over one hundred different variables. This invention results in a single solution in one bottle through all of these stages dramatically reducing the variables to less than an equivalent of ten.

It will be appreciated that the present invention uses a single media solution for culturing all the stages of embryonic growth and development. The desired formulation of this invention has been clearly defined and is based on the nutritional needs of oocytes, zygotes and embryos, through to blastocysts, from oocyte retrieval to blastocyst stage, to blastocyst implantation. This invention allows the implantation of the embryo at any stage of development. These stages can be immediately after an oocyte is fertilized, at the four-cell embryo stage, or at the eight-cell embryo or blastocyst stage.

A unique feature of the culture solution and its formulation is that it includes EDTA. This ingredient is only added to the conventional solutions within the second or later stages of development and is only added to conventional sequential media's for these later stages. This is a significant ingredient used in embryonic development and the fact that it is contained in the invention through all stages of development from the retrieval of the oocytes through the eight-cell blastocyst development stage of the embryo. Therefore the solution of the invention and by not requiring the change in type of other media solutions will be of the same and consistent osmolality and pH levels.

The solution of the invention improves the development of the oocytes through the embryonic stages and may contain several types of proteins. These protein sources may include human serum albumin derived from human blood plasma, a synthesized human serum albumin or a recombinant serum albumin. Recombinant serum albumin may be a desirable protein to use, as it may be more desirable because it may decrease the risk of impurities related to blood derived serum albumin. The solution may also contain a combination of ingredients, such as human serum albumin, human alpha-globulin and beta globulin additives mixed in a sterile saline solution. This may vary according to individual preferences.

The solution includes a unique protocol to include the use of the exact same solution and the exact same bottle of solution through the entire development stages of the embryo. This solution is superior to all other media and sequential media formulations in that it will allow the embryos to be cultured from a single solution and or bottle of the solution for the full one, two, three, four or five days of development, without subjecting the embryos at second solution which is a separate and different medial formulations, which may result or cause embryonic shock or any changes in osmolality or pH, or problems created by changes or additions to the solutions. The solution may be refreshed one or more times during the culturing protocol.

The solution may be used as the media solution in which the embryo is contained for the implantation of the embryo into the patient. The media solution may be supplemented with a concentration of hyaluronan and/or a combination of a concentration of hyaluronan and a recombinant human albumin.

The solution may also be used as a reimplantation or transfer media, and may be used in combination with a media solution which includes hyaluronan and a recombinant human albumin.

The solution must contain a base of sterile water; an effective amount of salts operative to support embryonic growth from an embryo formation stage to an embryo reimplantation stage; an effective amount of a buffer operative to maintain a solution pH of about 7.35 throughout the full embryonic growth from an embryo formation stage to an embryo reimplantation stage; an effective amount of energy substrates operative to support embryonic growth from an embryo formation stage to an embryo reimplantation stage; an effective amount of essential and nonessential amino acids operative to duplicate female reproductive fluids from an embryo formation stage to an embryo reimplantation stage; the glutamine dipeptide, glycyl-glutamine; an effective amount of a chelator operative to maintain the solution at a consistent pH and osmolality throughout the entire culturing stage from the embryo formation stage to the embryo reimplantation stage; a pH indicator; and an antibiotic.

Since many changes and variations of the disclosed embodiments of the invention may be made without departing from the inventive concept, it is not intended to limit the invention except as required by the appended claims.

What is claimed is:

1. A single embryo culturing solution which supports embryonic growth from an embryo formation stage to an embryo reimplantation stage, said solution containing:
a) a base of sterile water;
b) an effective amount of salts operative to support embryonic growth from an embryo formation stage to an embryo reimplantation stage;
c) an effective amount of a buffer operative to maintain a solution pH of about 7.35 throughout the full embryonic growth from an embryo formation stage to an embryo reimplantation stage;
d) an effective amount of energy substrates operative to support embryonic growth from an embryo formation stage to an embryo reimplantation stage;
e) an effective amount of essential and nonessential amino acids operative to duplicate female reproductive fluids from an embryo formation stage to an embryo reimplantation stage;
f) the glutamine dipeptide, glycyl-glutamine;
g) an effective amount of a chelator operative to maintain the solution at a consistent pH and osmolality throughout the entire culturing stage from the embryo formation stage to the embryo reimplantation stage;
h) a pH indicator; and
i) an antibiotic.

2. A method for the culturing of newly fertilized embryos from an embryo formation stage to an embryo reimplantation stage, said method comprising the steps of:
a) providing an embryo culturing solution comprising: a base of sterile water; an effective amount of salts operative to support embryonic growth from an embryo formation stage to an embryo reimplantation stage; an effective amount of a buffer operative to maintain a solution pH of about 7.35 throughout the full embryonic growth from an embryo formation stage to an embryo reimplantation stage; an effective amount of energy substrates operative to support embryonic growth from an embryo formation stage to an embryo reimplantation stage; an effective amount of essential and nonessential amino acids operative to duplicate female reproductive fluids from an embryo formation stage to an embryo reimplantation stage; the glutamine dipeptide, glycyl-glutamine; an effective amount of a chelator operative to maintain the solution at a consistent pH and osmolality throughout the entire culturing stage from the embryo formation stage to the embryo reimplantation stage; a pH indicator; and an antibiotic;
b) adding one or more newly fertilized stage embryos to said embryo culturing solution;
c) growing said one or more embryos in said embryo culturing solution from said newly fertilized embryo stage to second embryo reimplantation stage embryos; and
d) removing said second reimplantation stage embryos from said embryo culturing solution.

3. A method of forming an embryo culturing solution which is used in culturing of newly fertilized embryos from an embryo formation stage to an embryo reimplantation stage, said method comprising the steps of:
a) providing a sterile water base;
b) adding an effective amount of salts operative to support embryonic growth from an embryo formation stage to an embryo reimplantation stage to said water base;
c) adding an effective amount of a buffer operative to maintain a solution pH of about 7.35 throughout the full embryonic growth from an embryo formation stage to an embryo reimplantation stage to said water base;
d) adding an effective amount of energy substrates operative to support embryonic growth from an embryo formation stage to an embryo reimplantation stage to said water base;
e) adding an effective amount of essential and nonessential amino acids operative to duplicate female reproductive fluids from an embryo formation stage to an embryo reimplantation stage;
f) adding the glutamine dipeptide, glycyl-glutamine;
g) adding an effective amount of a chelator operative to maintain the solution at a consistent pH and osmolality throughout the entire culturing stage from the embryo formation stage to the embryo reimplantation stage;
h) adding a pH indicator; and
i) adding an antibiotic.
